(19) United States
(12) Patent Application Publication
(10) Pub. No.: US 2025/0339572 A1
(43) Pub. Date: Nov. 6, 2025

(54) Solution Loaded with Alpha-Emitter Radionuclides

(71) Applicant: ALPHA TAU MEDICAL LTD., Jerusalem (IL)

(72) Inventors: Vered Domankevich, Hod Hasharon (IL); Michael Schmidt, Kfar Saba (IL); Itzhak Kelson, Tel Aviv (IL); Lior Arazi, Tel Aviv (IL); Ronen Segal, Jerusalem (IL); Amnon Gat, Matan (IL); Dor Levy, Petach Tikva (IL)

(21) Appl. No.: 19/269,157

(22) Filed: Jul. 15, 2025

Related U.S. Application Data
(63) Continuation-in-part of application No. PCT/IB2024/050380, filed on Jan. 15, 2024.
(60) Provisional application No. 63/480,016, filed on Jan. 16, 2023.
(60) Provisional application No. 63/450,971, filed on Mar. 9, 2023.
(60) Provisional application No. 63/671,825, filed on Jul. 16, 2024.

(51) Int. Cl. A61K 51/12; A61K 9/107; A61K 31/7105; A61K 31/711; A61K 35/74; A61K 45/06; A61K 47/44

(52) U.S. Cl. CPC A61K 51/1217; A61K 9/107; A61K 31/7105; A61K 31/711; A61K 35/74; A61K 45/06; A61K 47/44

(57) ABSTRACT

A method of generating an emulsion for treating a patient. The method includes generating a water-based solution carrying an agent which turns into a hydrogel by addition of calcium ions. Radium radionuclides are added to the water-based solution in a concentration sufficient for radiotherapy. After adding the radium to the water-based solution, the water-based solution is mixed with an emulsifier and an oil.

### CROSS-REFERENCE TO RELATED APPLICATIONS

This application claims the benefit of U.S. provisional application 63/671,825, filed Jul. 16, 2024. This application is also a continuation in part of PCT application PCT/IB2024/050380, which claims the benefit of U.S. provisional application 63/480,016, filed Jan. 16, 2023, and U.S. provisional application 63/450,971, filed Mar. 9, 2023. These applications are incorporated herein by reference in their entirety.

### FIELD OF THE INVENTION

The present invention relates generally to tumor therapy and particularly to intra-tumoral alpha-emitter radiation therapy.

### BACKGROUND OF THE INVENTION

Ionizing radiation is commonly used in the treatment of certain types of tumors, including malignant cancerous tumors, to destroy their cells. Alpha radiation is one of the most powerful radiation types for cell destruction, but its range is very short and therefore its delivery to tumors is a challenge.

Diffusing alpha-emitters radiation therapy (DaRT), described for example in U.S. Pat. No. 8,834,837 to Kelson, mounts alpha-emitting radium radionuclides on a source (also referred to as a seed) in a manner that the radium radionuclides generally do not leave the source, but a substantial percentage of their daughter radionuclides (radon-220 in the case of radium-224 and radon-219 in the case of radium-223) leave the source into the tumor, upon radium decay. These radionuclides, and their own radioactive daughter atoms, spread around the source by diffusion up to a radial distance of a few millimeters before they decay by alpha emission. Thus, the range of destruction in the tumor is increased relative to radionuclides which remain with their daughters on the source.

In order for the treatment of a tumor to be effective, DaRT seeds employed in the treatment should be implanted throughout the tumor at small distances, e.g., less than 5 millimeters, from each other. Some tumors are easily accessible externally by a physician for implantation of the seeds, while other tumors are in internal organs. In addition, seed implantation in some organs may cause suffering and pain such as in the case of the eyeball. Even for easily accessible tumors, the destruction of a tumor may require implantation of over one hundred seeds. In addition, production of the seeds is a demanding and expensive task.

Another method used to deliver alpha emitting radioactive atoms to malignant cells is targeted radionuclide therapy using methods such as radioimmunoconjugates. In targeted therapy, targeting-carriers, such as antibodies and/or liposomes, are connected to radioactive atoms and injected into the blood stream of a patient. During circulation, the targeting-carriers attach to or remain next to malignant cells and when alpha particles are emitted by the radioactive atoms at least some of the emitted alpha particles destroy the malignant cells.

PCT publication WO01/60417 to Larsen, titled “Radioactive Therapeutic Liposomes”, PCT publication WO 02/05859 to Larsen, titled: “Method of Radiotherapy” and US patent publication 2004/0208821 to Larsen, titled: “Method of Radiotherapy”, the disclosures of which are incorporated herein by reference in their entirety, describe liposomes which encapsulate heavy radionuclides which emit alpha particles. The radionuclides may include, among others, Radium-223, Radium-224 and Thorium-227. Daughter radionuclides generally remain trapped during nuclear transformation of the radionuclides.

PCT publication WO2006/110889, titled: “Multi-Layer Structure having a Predetermined Layer Pattern Including an Agent”, describes a polymer multilayer structure which can be used to deliver radioisotopes for radiotherapy.

Alpha emitting radioactive atoms may also be delivered to a tumor in microparticles or nanoparticles which limit the transport of the radioactive atoms into the blood stream and away from the tumor. US patent publication 2017/0000911, titled “Radiotherapeutic Particles and Suspensions”, states that the microparticles and nanoparticles may be stable or slowly degrading.

PCT publication WO2010/028048, titled: “Brachytherapy Seed with Fast Dissolving Matrix for Optimal Delivery of radionuclides to Cancer Tissue”, describes polymer seeds embedding in them microspheres containing beta-emitting or alpha emitting radionuclides. After the seeds are implanted in a tumor, they are dissolved so that the radionuclides in the microspheres can destroy tumor cells.

U.S. Pat. No. 8,470,294 describes flexible brachytherapy seeds.

U.S. Pat. No. 9,539,346 to Larsen et al. and the paper Westrøm S, Malenge M, Jorstad I S, Napoli E, Bruland Ø S, Bønsdorff T B, Larsen R K, “Ra-224 labeling of calcium carbonate microparticles for internal α-therapy: Preparation, stability, and biodistribution in mice”, J Labelled Comp Radiopharin. 2018 May 30; 61(6):472-486, doi: 10.1002/jlcr.3610. Epub 2018 Mar. 12. PMID: 29380410; PMCID: PMC6001669, propose use of calcium carbonate microparticles as carriers for radium-224, designed for local therapy of disseminated cancers in cavitary regions.

US patent publication 2022/0152228 to Thorek et al., describes administration of an alpha particle emitting therapeutic agent chelating a radiotherapeutic agent with a macrocyclic molecule or combined with an ion-modulating agent, in an injectable gel or hydrogel.

U.S. Pat. No. 7,776,310 to Kaplan, titled: “Flexible and/or Elastic Brachytherapy Seed or Strand”, describes a brachytherapy strand that is elastic and/or flexible and preferably biodegradable, which carries radionuclides which may emit gamma, beta or alpha radiation. In one embodiment, the strands are hydrogel strands prepared by dripping a polymer solution, such as alginate, from a reservoir though microdroplet forming device into a stirred ionic bath.

A paper titled “Preparation of a radionuclide/gel formulation for localised radiotherapy to a wide range of organs and tissues”, by Holte et al., Pharmazie 61 (2006), describes encapsulating radio labelled particles into a gel formulation, including alginate gels.

A paper by Yu Chao et al., titled “Combined local immunostimulatory radioisotope therapy and systemic immune checkpoint blockade imparts potent antitumor responses”, Nature Biomedical Engineering, Vol. 2, August 2018, describes use of a radio-isotope-labelled natural enzyme (¹³¹I-Cat), a natural polysaccharide alginate and synthetic oligodeoxynucleotides CpG for cancer treatment.

The book “In-Situ Gelling Polymers: for Biomedical Applications” mentions various hydrogels which are used in slow release of drugs.

PCT publication 2023/066994 titled “Peptide-coupled Alginate Gels comprising Radionuclides”, describes use of a peptide-coupled alginate gel in radiotherapy.

### SUMMARY OF THE INVENTION

There is therefore provided in accordance with embodiments of the present invention, a method of generating an emulsion for treating a patient, comprising generating a water-based solution carrying an agent which turns into a hydrogel by addition of calcium ions, adding radium radionuclides to the water-based solution in a concentration sufficient for radiotherapy; and mixing the water-based solution with an emulsifier and an oil, after adding the radium to the water-based solution.

Optionally, the emulsifier with the oil are of a volume greater than the volume of the water-based solution. Optionally, the agent which turns into a hydrogel by addition of calcium ions is between 0.5-4% of the water-based solution. Optionally, the agent which turns into a hydrogel by addition of calcium ions is between 1.2-2.4% of the water-based solution. Optionally, the emulsifier and oil form less than 80% of the emulsion. Optionally, the emulsifier and oil form at least 75% of the emulsion. Optionally, the emulsifier with the oil is Incomplete Freund's Adjuvant (IFA) or Complete Freund's Adjuvant (CFA). Optionally, the emulsion does not include targeting elements which make the emulsion suitable for targeted therapy. Optionally, the method includes adding to the water-based solution a substance which regulates immune-checkpoints. Optionally, the method includes adding to the water-based solution an immunoadjuvant. In some embodiments, the method includes adding a mycobacterium to the water-based solution. Optionally, the method includes adding a DNA or RNA viral mimic to the water-based solution. Alternatively or additionally, the method includes adding a tumor antigen to the water-based solution. In some embodiments, the agent comprises alginate. Optionally, the radium radionuclides are radium-224 radionuclides. Optionally, the emulsion: does not include calcium; or does not include a sufficient amount of calcium for turning the agent into a hydrogel. Optionally, generating the water-based solution comprises dispersing the agent homogenously in the water-based solution. In some embodiments, mixing the water-based solution with an emulsifier and an oil is performed in a syringe suitable for injecting the emulsion to the patient.

There is further provided in accordance with embodiments of the present invention, a medicament for treating a tumor, comprising an agent which turns into a hydrogel by addition of calcium ions, a water-based solution carrying the agent in a manner allowing injection of the medicament into a patient, an emulsifier, an oil and radium radionuclides bounded to the agent in a concentration sufficient to treat the tumor by radiotherapy. Optionally, the agent is dispersed homogenously in the water-based solution. Optionally, the emulsifier and oil are Incomplete Freund's Adjuvant (IFA) or Complete Freund's Adjuvant (CFA). Optionally, the medicament does not include targeting elements sufficient for targeted therapy of the medicament. Optionally, the medicament includes a substance which regulates immune-checkpoints dispersed in the medicament. Alternatively or additionally, the medicament includes an immunoadjuvant.

There is further provided in accordance with embodiments of the present invention, a mixture for treating a tumor, comprising an agent which turns into a hydrogel by addition of calcium ions, a vehicle carrying the agent and radium radionuclides bounded to the agent in a concentration sufficient to treat the tumor by radiotherapy. The vehicle optionally carries the agent in a manner allowing administration of the mixture to the patient, for example by injection of the mixture into the patient (e.g., into the tumor), and/or by smearing on a tumor or tumor remnants.

Optionally, the mixture has a viscosity of less than 20,000 cP. Optionally, the vehicle comprises an aqueous solution, and the agent is dispersed homogenously in the aqueous solution. Optionally, the mixture further includes a substance which regulates immune-checkpoints dispersed in the mixture and/or a contrast material. In some embodiments, the mixture has a viscosity suitable for injection through a needle into a tumor. Optionally, the mixture is thermosensitive such that the viscosity of the mixture increases by at least a factor of two when its temperature increases from room temperature to body temperature. Optionally, the mixture is adapted to solidify less than two hours from being injected into the tumor. In some embodiments, the agent comprises alginate and/or Pluronics. In some embodiments, the radium radionuclides are radium-224 radionuclides. Optionally, the mixture further comprises calcium. Optionally, the mixture comprises calcium at a concentration of between about 5-10 millimolar. In some embodiments, the mixture does not bond to radon and lead. Optionally, the agent which turns into a hydrogel by addition of calcium ions is between 2-4% of the mixture.

There is further provided in accordance with embodiments of the present invention, a method for treating a tumor, comprising injecting into the tumor a mixture including an agent which turns into a hydrogel in contact with calcium ions; and injecting into the tumor radium radionuclides of an activity suitable for treatment of the tumor. In some embodiments, the method includes injecting calcium into the tumor. Optionally, injecting the calcium into the tumor is performed before injecting the mixture. Alternatively or additionally, injecting the calcium into the tumor is performed after injecting the mixture into the tumor. In some embodiments, injecting the calcium into the tumor comprises including calcium in the mixture and injecting the mixture into the tumor. Optionally, injecting the calcium into the tumor comprises concurrently injecting the mixture and the calcium. In some embodiments, injecting the radium radionuclides into the tumor comprises including radium in the mixture and injecting the mixture into the tumor.

There is further provided in accordance with embodiments of the present invention, a method for generating a mixture for treating a tumor, comprising providing an inert excipient, adding an agent which turns into a hydrogel in contact with calcium ions to the inert excipient; and adding radium radionuclides in a concentration sufficient to treat the tumor by radiotherapy, to the inert excipient.

Optionally, adding the agent and the radium to the inert excipient comprises combining the agent and the radium before they are added to the inert excipient, and adding the combination of the agent and the radium together to the inert excipient. Alternatively or additionally, adding the agent and the radium to the inert excipient comprises adding the radium to the inert excipient only after the agent is added to the inert excipient.

There is further provided in accordance with embodiments of the present invention, a mixture for treating a tumor, comprising an agent which turns into a hydrogel by addition of calcium ions, a vehicle carrying the agent in a manner allowing injection of the mixture into a patient, small particles dispersed in the vehicle; and radium radionuclides on or in the small particles in a concentration sufficient to treat the tumor by radiotherapy. Optionally, the small particles comprise nanoparticles.

There is further provided in accordance with embodiments of the present invention, a mixture for treating a tumor, comprising an agent which turns into a hydrogel by addition of calcium ions, a vehicle carrying the agent in a manner allowing injection of the mixture into a patient, calcium sulfate at a concentration of between 1-10 millimolar; and radium radionuclides bounded to the agent in a concentration sufficient to treat the tumor by radiotherapy.

There is further provided in accordance with embodiments of the present invention, a mixture for treating a tumor, comprising an agent which turns into a hydrogel by addition of calcium ions, a vehicle carrying the agent in a manner allowing injection of the mixture into a patient, barium or magnesium at a concentration of between 1-10 millimolar; and radium radionuclides bounded to the agent in a concentration sufficient to treat the tumor by radiotherapy.

There is further provided in accordance with embodiments of the present invention, a mixture for treating a tumor, comprising an agent which turns into a hydrogel by addition of calcium ions, a vehicle carrying the agent in a manner allowing injection of the mixture into a patient, an Ethylenediaminetetraacetic acid (EDTA) and/or dodecane tetraacetic acid (DOTA) chelator; and radium radionuclides bounded to the agent in a concentration sufficient to treat the tumor by radiotherapy.

There is further provided in accordance with embodiments of the present invention, a mixture for treating a tumor, comprising an agent which turns into a hydrogel by addition of calcium ions, a vehicle carrying the agent in a manner allowing injection of the mixture into a patient, disodium phosphate, gelatin, Chitosan, and/or polymer phosphate; and radium radionuclides bounded to the agent in a concentration sufficient to treat the tumor by radiotherapy.

Optionally, the vehicle comprises an aqueous solution, and the agent is dispersed homogenously in the aqueous solution. The mixture optionally further includes a substance which regulates immune-checkpoints dispersed in the mixture and/or a contrast material. Optionally, the mixture is thermosensitive such that the viscosity of the mixture increases by at least a factor of two when its temperature increases from room temperature to body temperature. Optionally, the agent comprises alginate and/or Pluronics. Optionally, the radium radionuclides are radium-224 radionuclides. In some embodiments, the mixture further comprises calcium. Optionally, the mixture comprises calcium at a concentration of between 1-10 millimolar. Optionally, the mixture does not bond to radon and lead. Optionally, the agent which turns into a hydrogel by addition of calcium ions is between 0.5-4% of the mixture. Optionally, the agent is included in small particles carried by the vehicle. Optionally, the small particles comprise a metallic core surrounded by the agent.

There is further provided in accordance with embodiments of the present invention, a method for treating a tumor, comprising injecting, into a patient, at a first time, a mixture including an agent which turns into a hydrogel in contact with calcium ions; and injecting into the patient, at a second time, radium radionuclides of an activity suitable for treatment of the tumor and calcium for hardening the agent. Optionally, injecting the radium and calcium at the second time is performed at least 1 hour after injecting the mixture at the first time. Optionally, injecting the radium and calcium at the second time is performed at least 4 hours after injecting the mixture at the first time.

There is further provided in accordance with embodiments of the present invention, a method for treating a tumor, comprising administering, into a patient, flexible or solid seeds formed from a mixture including an agent which turns into a hydrogel in contact with calcium ions, wherein the seeds carry radium radionuclides of an activity suitable for treatment of the tumor and calcium for hardening the agent, wherein the seeds have a width of at least 100 microns. Optionally, administering the seeds comprises inserting a needle carrying the seeds into the patient and retracting the needle in a manner leaving the seeds in the patient while removing the needle from the patient. Optionally, administering the seeds comprises placing the seeds on a surface requiring treatment. Optionally, administering the seeds comprises placing the seeds in a cavity formed by removal of a tumor.

There is further provided in accordance with embodiments of the present invention, a method for treating a tumor, comprising injecting, into a patient, at a first time, a mixture including an agent which turns into a hydrogel in contact with calcium ions, injecting into the patient, at a second time and a third time, calcium for hardening the agent; and providing radium radionuclides of an activity suitable for treatment of the tumor in the mixture, wherein the radium is included in the mixture at the first time or is introduced into the mixture after the first time. Optionally, injecting the calcium into the tumor at the second time is performed before injecting the mixture at the first time. Optionally, injecting the calcium into the tumor at the second time is performed after injecting the mixture into the tumor at the first time.

There is further provided in accordance with embodiments of the present invention, a method for generating a mixture for treating a tumor, comprising determining a type of the tumor to be treated, providing an inert excipient, adding an agent which turns into a hydrogel in contact with calcium ions to the inert excipient, adding an amount of calcium to the mixture, wherein the amount of calcium is selected responsive to the determined type of the tumor; and adding radium radionuclides in a concentration sufficient to treat the tumor by radiotherapy, to the inert excipient. Optionally, adding the agent and the radium to the inert excipient comprises combining the agent and the radium before they are added to the inert excipient, and adding the combination of the agent and the radium together to the inert excipient. Optionally, adding the agent and the radium to the inert excipient comprises adding the radium to the inert excipient only after the agent is added to the inert excipient.

There is further provided in accordance with embodiments of the present invention, a method for treating a tumor, comprising administering, into a cavity in a patient, a mixture including an agent which turns into a hydrogel in contact with calcium ions; injecting into the cavity radium radionuclides of an activity suitable for treatment of the tumor; and expanding a balloon in the in a manner which presses the mixture and radium against a wall of the cavity.

The method optionally further includes administering to the mixture in the cavity a substance which causes the mixture to harden or gelate. The method optionally further includes removing the balloon from the cavity after the mixture hardened or gelated. Optionally, the substance which causes the mixture to harden or gelate comprises calcium.

There is further provided in accordance with embodiments of the present invention, a method for treating a patient, comprising placing a polymer bag on a surface to be treated; and injecting into the polymer bag a mixture including an agent which turns into a hydrogel in contact with calcium ions and radium radionuclides of an activity suitable for treatment of the tumor. Optionally, the polymer bag has a thick side having a thickness of more than 100 microns and a thin side having a thickness of less than 100 microns, and wherein the thin side of the bag is placed on the surface to be treated.

### BRIEF DESCRIPTION OF THE DRAWINGS

FIG. 1 is a flowchart of acts performed in producing a mixture for delivery of radium to a tumor, in accordance with an embodiment of the present invention;

FIG. 2 is a flowchart of acts performed in treatment of a tumor, in accordance with an embodiment of the present invention;

FIGS. 3A and 3B are graphs showing results of an experiment of a treatment, in accordance with an embodiment of the present invention; and

FIGS. 4A and 4B are further graphs showing results of an experiment of a treatment, in accordance with an embodiment of the present invention.

### DETAILED DESCRIPTION OF EMBODIMENTS

An aspect of some embodiments of the invention relates to delivering alpha-emitter radium radionuclides to a tumor, tumor bed, or other site requiring treatment, in a mixture (e.g., solution) which comprises an agent which turns into a hydrogel by addition of calcium ions. In some embodiments, the mixture comprises calcium ions and/or other ions with properties similar to calcium, which can turn the liquid mixture into a hydrogel. The mixture serves as a medicament administered to a patient for treatment of unwanted cells, such as cancerous cells. The calcium and/or other ions optionally crosslink polymer chains of the agent to form a polymeric net. This may occur, for example, in a manner similar to the “egg-box” model described in “A Study of Sodium Alginate and Calcium Chloride Interaction Through Films for Intervertebral Disc Regeneration Uses”, Congresso Brasileiro de Engenharia e Ciência dos Materiais 09 a 13 de Novembro de 2014, the disclosure of which is incorporated herein by reference.

Use of such a mixture has the advantage that the agent chemically bounds to the radium, which is similar in some chemical properties to calcium, basically preventing the radium from leaving the mixture, while the mixture remains in the tumor for a sufficient time required for the treatment, due to gelation. On the other hand, the agent does not substantially bound to descendant radionuclides of the radium, such as radon and lead, which are allowed to diffuse or otherwise disperse throughout a tumor in which the mixture is implanted, despite the presence of the mixture in the tumor. The mixture is optionally injectable.

### Composition

The mixture optionally comprises in a vehicle, an agent which turns into a hydrogel when forming contact with calcium ions, and radium radionuclides which couple to the agent. In some embodiments, the vehicle comprises an inert excipient, such as water, saline and/or phosphate-buffered saline (PBS). In some embodiments, the mixture includes one or more additional drugs which are to be delivered with the radionuclides. In some embodiments, the mixture includes one or more other materials, such as a contrast material or isotope used for imaging.

The mixture optionally comprises a biocompatible aquatic solution, which has a viscosity suitable for direct injection into a tumor. The aquatic solution optionally has a viscosity of at least 10, 20, 50 or even 200 centipoise (cP), but lower than 1,000 cP, at 20 degrees Celsius. Alternatively, the aquatic solution has a high viscosity of at least 2,000 cP, at least 5,000 cP, or even at least 10,000 cP. Optionally, the required viscosity is achieved by adding a sufficient amount of calcium to the mixture. Generally, the more calcium added to the mixture, the higher the viscosity of the mixture. Alternatively, other materials may be added to the mixture to control its viscosity.

In some embodiments, the mixture is designed to spread within a tumor to which it is delivered, but to be sufficiently viscous to remain in the tumor and hold the radium within the tumor until at least 80%, at least 90%, at least 95% or even at least 97% of the radium radionuclides have undergone radioactive decay. Optionally, after injection, the mixture is sufficiently viscous such that 24 hours after delivery into a tumor, not more than 50%, not more than 30%, not more than 10%, not more than 5% or even not more than 3% of the agent, leaves the tumor.

In some embodiments, the components of the mixture that hold the radium in the tumor are not biodegradable, or are biodegradable but are only slowly degradable or begin to degrade only a predetermined period after injection, such that not more than 80%, not more than 50%, not more than 40%, not more than 25%, not more than 15%, not more than 5%, not more than 3% or even not more than 1% of the radium that did not undergo radioactive decay is allowed to escape the tumor.

Alternatively or additionally, part of the components of the mixture not required to hold the radium in the tumor, are biodegradable in order to allow for closer and direct contact between the radium and tumor cells.

### Preparation

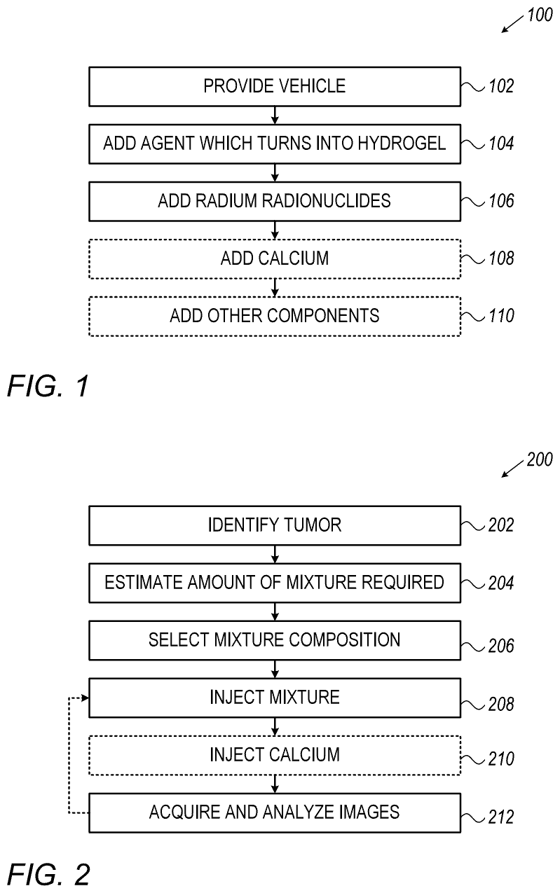

FIG. 1 is a flowchart of acts performed in producing a mixture for delivery of radium to a tumor, in accordance with an embodiment of the present invention. The method (100) includes providing (102) a vehicle, and adding (104) to the vehicle, the agent which turns into a hydrogel when forming contact with calcium ions. In some embodiments, the vehicle serves as a diluter of the agent. Alternatively or additionally, small particles which comprise the agent are dispersed in the vehicle. Radionuclides of radium are added (106) to the agent, before or after the agent is added to the vehicle. In some embodiments, calcium is added (108) to the mixture. Alternatively or additionally, other components are added (110) to the mixture, such as one or more therapeutic drugs, an in situ gelling polymer and/or contrast materials. These other components are added to the vehicle before adding the agent to the vehicle, or they are added to the mixture after adding the agent to the vehicle. In some embodiments, some or all of the components of the mixture are each provided in a separate solution, and the solutions are combined to form the mixture. In some embodiments, a first solution of sodium alginate (also known as alginate), for example, 10% sodium alginate, and a second solution of radium-224 radionuclides are prepared separately. The first and second solution optionally have the same size (e.g., 100 microliter each). The first and second solutions are then mixed together, such that the alginate concentration goes down, e.g., to 5%.

It is noted that the method of FIG. 1 is just one example of the methods that may be used to create the mixture. Particularly, the components of the mixture may be combined in any suitable order. For example, in other embodiments, the radium is added to the vehicle before the agent is added to the vehicle. In some embodiments, the calcium is added to the vehicle before the radium and/or the agent. In other embodiments, the agent and calcium are mixed, or an agent solution and calcium solution are mixed together, and radium is added only thereafter. In still other embodiments, the calcium and radium, or solutions thereof, are mixed together, and the combined radium and calcium are mixed with the agent. Optionally, after adding the radium to the mixture, the mixture is left for an incubating period of at least 45 seconds, at least 90 seconds, at least 210 seconds, at least 360 seconds or even at least 10 minutes, in which the radium is allowed to spread and/or couple to the agent. In some embodiments the mixture is stirred or otherwise mixed to achieve a more homogeneous spread of its components. In some embodiments the mixture is sterilized before delivery to the tumor, for example using autoclave sterilization. Optionally, the sterilization is performed after the components are mixed. Alternatively, the components are sterilized separately. The mixing is optionally performed at room temperature.

In some embodiments, the mixture is prepared by adding radium radionuclides to an alginate based product available commercially, such as GUARDIX® SG.

### The Agent

Referring in more detail to adding (104) the agent, the agent optionally comprises sodium alginate, such as described in Abasalizadeh, F., Moghaddam, S. V., Alizadeh, E, et al. Alginate-based hydrogels as drug delivery vehicles in cancer treatment and their applications in wound dressing and 3D biopriming. J Biol Eng 14, 8 (2020), https://doi.org/10.1186/s13036-020-0227-7, and/or PCT publication WO2019/043699, which are incorporated herein by reference. The term alginate refers herein to sodium alginate and/or to esters, salts and other derivatives thereof.

The alginate optionally has a high guluronic acid to mannuronic acid ratio (G/M ratio) of at least 1.5 or even at least 1.8, to increase the capability of the alginate to capture the calcium and/or radium. It is noted, however, that in some embodiments the alginate has a low G/M ratio of less than 2, less than 1.5 or even less than 1.

In some embodiments, the alginate has a molecular weight (MW) lower than 75 kDa (e.g., FMC Biopolymers (Drammen, Norway) PRONOVA UP VLVG alginate or PRONOVA UP VLVM alginate). In other embodiments, the alginate has a MW in the range of 75-200 kDa (e.g., FMC Biopolymers (Drammen, Norway) PRONOVA UP LVG alginate, FMC Biopolymers (Drammen, Norway) PRONOVA UP LVM alginate, PRONOVA SLM₂₀, PRONOVA SLG₂₀). In still other embodiments, the alginate has a MW in the range of 150-250 kDa (e.g., PRONOVA SLM₁₀₀, PRONOVA SLG₁₀₀) In further embodiments, the alginate has a MW above 200 kDa (e.g., FMC Biopolymers (Drammen, Norway) PRONOVA UP MVG alginate, FMC Biopolymers (Drammen, Norway) PRONOVA UP MVM alginate). The use of a high molecular weight is particularly preferred when a high viscosity is desired to prevent leakage of the mixture from the tumor. Optionally, higher molecular weight of the alginate is used when the concentration of the alginate in the mixture is relatively low.

The alginate may be ultrapure, sterile, and/or peptide coupled (e.g., GRGDSP-coupled, VAPG-coupled, REDV-coupled).

Alternatively or additionally, the agent comprises poloxamer, also known as Pluronics.

Optionally, the agent is not biodegradable, or is slowly biodegradable such that substantial degradation begins only after at least one, at least 2 or even after at least 5 half-lives of the radium radionuclides. For radium-224, the substantial degradation of the agent optionally begins only after at least 10 days, at least 20 days or even at least a month from implantation. The degradation to a level of less than 10% optionally continues over a period of at least 10 days, at least 20 days or even at least 30 days. Thus, the agent fixes the radium in place as long as it has substantial radioactivity.

In some embodiments, the agent is at least 0.1%, at least 0.4%, at least 0.5%, at least 1%, at least 2%, at least 3.5%, at least 4.5%, at least 5%, at least 6.5% or even at least 8% of the mixture in weight/weight. In some embodiments, the agent is less than 12%, less than 10%, less than 8%, less than 6.5%, less than 5%, less than 4% or even less than 3% of the mixture in weight/weight. In particular embodiments, solutions in which the agent was 2%, 4% and 5% were used, although other concentrations of the agent are also contemplated.

In embodiments in which the mixture includes low levels of calcium (e.g., less than 3 or even less than 2 milli-molar) in the mixture or even no calcium at all, such that the gelation is mainly due to endogenous calcium and/or other ions, the agent is optionally included in the mixture in a concentration of at least 3 mg/ml, or even at least 5 mg/ml. Optionally, the agent is included in a concentration of less than 7 mg/ml or even less than 6 mg/ml so that the mixture assumes a weak mechanical strength gel, which semi-uniformly disperses in the tumor, so that the radium in the mixture is distributed throughout the tumor, without substantial leakage outside of the tumor.

In some embodiments, the agent is included in a concentration of at least 5 mg/ml, 7 mg/ml, at least 9 mg/ml or even at least 12 mg/ml so that the mixture assumes a more viscous solid and greater-mechanical-strength gel form.

For a mixture with higher levels of calcium, such as more than 4 or even more than 5 milli-molar, the agent concentration is optionally less than 6 mg/ml, less than 5 mg/ml, less than 4 mg/ml or even less than 2 mg/ml.

In some embodiments, the agent is dispersed homogeneously in the vehicle. Accordingly, the radium, which couples to the agent, is dispersed homogeneously in the mixture. In other embodiments, the mixture is non-homogenous, including small particles (e.g., microparticles, nanoparticles and/or beads) which are formed from the agent, carry the agent or carry at least a substantial percentage of the agent. The non-homogenous structure increases the surface area of the agent from which radon can escape. As the non-agent portions of the mixture dissolve, the uniformity of the spread of the agent to which the radium is bounded increases. In still other embodiments, some (e.g. at least 20%, at least 40% or even at least 60%) of the agent in the mixture is dispersed throughout the vehicle, while another portion of the agent (e.g. at least 25%, at least 35% or at least 45%) is included in small particles.

In the present application, the term “microparticles” refers to particles having a diameter of between 0.1 and 100 micrometers. The term “nanoparticles” refers to particles having a diameter of between 0.1 and 100 nanometers. It is noted that in some embodiments the small particles are larger than microparticles for example having a diameter of at least 120 micrometers, at least 150 micrometers, at least 180 micrometers, at least 250 micrometers or even at least 400 micrometers. Optionally, in these embodiments, the small particles have a diameter of less than 500 micrometers. In some embodiments, the small particles are spheres and/or beads. Alternatively, the small particles are of any other suitable shape.

In some embodiments, the small particles are formed by dripping a solution of the agent into a high-concentration calcium solution. In other embodiments, the small particles are produced by adding air to a solution of the agent and calcium. Alternatively or additionally, the small particles are formed by mixing the agent along with an aquatic solution. In some embodiments, the small particles are created by changing the relation between the agent and the calcium such that the resultant mixture includes small particles in an aquatic solution. The small particles are produced using any suitable method known in the art, such as any of the methods in Andrea Dodero et al., “An Up-To-Date Review on Alginate Nanoparticles and Nanofibers for Biomedical and Pharmaceutical Applications”, Advanced Materials Interfaces, Vol. 8, Issue 22, Nov. 23, 2021, Patricia Severino, “Alginate Nanoparticles for Drug Delivery and Targeting”, Current Pharmaceutical Design, Volume 25, issue 11, 2019, Anna Letocha, “Preparation and Characteristics of Alginate Microparticles for food, Pharmaceutical and Cosmetic Application”, Polymers 2022, and/or Jerome P. Paques, “Formation of Alginate nonospheres”, Thesis, Wageningen University, 2014.

The small particles may be of any suitable type known in the art, such as microfluidic devices, e.g. those provided by CD Bioparticles (https://www.cd-bioparticles.net/alginates), Elve Flow (https://www.elveflow.com/microfluidics-application-packs/nanoparticles-packs/easy-microfluidic-alginate-beads-generation-pack) and/or Thomas Scientific (https://www.thomassci.com/nav/cat1/alginatechitinbeads/0). Methods for production of small particles, which may be used in the present invention, are described, for example, in Alginate particle production, by Elve Flow http://www.techusci.com/UploadFiles/2021-03/369/2021032014063196895.pdf and/or Alginate Microbeads Production, by Fluigent (https://www.fluigent.com/wp-content/uploads/2022/01/alginate-beads-production-application-note.pdf).

In some embodiments, the small particles are formed from a solution which already includes the radium radionuclides. In other embodiments, the small particles are formed from an inert solution which does not include the radium radionuclides and the radium radionuclides are added to the small particles after their production, for example by incubation of the small particles in a radium solution.

Optionally, the small particles are formed such that the agent and bounded radium are on the outer surface of the small particles or close to the outer surface, in a manner that daughter radionuclides resulting from radioactive decay have a high probability, e.g., at least 25%, at least 35% or even at least 40%, of leaving the small particle. For example, when adding the radium to the small particles after their production, the addition is optionally performed such that the radium concentrates on the outer surfaces of the small particles, and not in their interior. Alternatively or additionally, the small particles are produced such that their interior is formed of a material which does not attract radium, while their outer surface includes a material which attracts radium. Alternatively, in some or all of the small particles, particularly nanoparticles which are small enough to allow diffusion of radon out of the nanoparticles even from their interior, the agent and radium are in the interior of the microparticle.

The small particles optionally have a viscosity of at least 100 cP, at least 200 cP, at least 300 cP or even at least 500 cP. Optionally, the viscosity of the particles is lower than 2,000 cP, lower than 1,500 cP or even lower than 1,000 cP.

### Radium

In some embodiments, the radium ions are not coupled to large particles that are made from materials other than the agent. Specifically, the radionuclides of radium are optionally not coupled to particles having a diameter greater than 100 nanometers, except for the agent, not coupled to targeting elements (e.g., antibodies) which couple to cells, not coupled to proteins and/or enzymes (e.g., catalase) and/or not coupled to vectors which are internalized into cancer cells (e.g., liposomes, radionucleosides). Optionally, the mixture does not include large microparticles, enzymes, liposomes, radionucleosides and/or targeting elements, which may couple to the radium. Thus, the movement of the radium is constrained by the agent, which reducing the percentage of the radium that leaves a tumor in which it is located, but may allow small movements within the tumor, in a manner achieving better coverage of the tumor by the radiation from the radium. In other embodiments, however, the radium is coupled to large particles and the dispersion of the radium throughout the tumor is achieved by the initial injection of the mixture throughout the tumor. In some of these other embodiments, the radium is mounted on, or contained in, microparticles or nanoparticles, e.g., of a metal base, that do not include the agent, and these microparticles or nanoparticles are placed in a mixture formed of the agent. The mixture serves, in these embodiments, to carry the microparticles and to collect radium that is released from the microparticles or nanoparticles. The microparticles or nanoparticles optionally have a manganese oxide outer surface which links to the radium. Alternatively or additionally, microparticles or nanoparticles are made of gold or have an outer gold surface which links to the radium. In still other embodiments, the agent, or the microparticles or nanoparticles include anti bodies linked to a chelator of radium.

The radium optionally includes radium-224 or radium-223. In some embodiments, the mixture includes at least 2 radium molecules per 1×10¹⁰ agent molecules, at least 5 radium molecules per 1×10¹⁰ agent molecules, at least 10 radium molecules per 1×10¹⁰ agent molecules, or even at least 20 radium molecules per 1×10¹⁰ agent molecules. Optionally, however, the mixture includes less than 100 radium molecules per 1×10¹⁰ agent molecules, less than 50 radium molecules per 1×10¹⁰ agent molecules, less than 30 radium molecules per 1×10¹⁰ agent molecules, or even less than 20 radium molecules per 1×10¹⁰ agent molecules. The number of agent molecules coupled to radium molecules is optionally less than 1%, less than 0.1%, less than 0.01% or even less than 0.001%.

The radium activity per volume is optionally at least 25 kilobecquerel per milliliter of the mixture, at least 75 kilobecquerel per milliliter of the mixture, at least 150 kilobecquerel per milliliter of the mixture, at least 300 kilobecquerel per milliliter of the mixture, or even at least 500 kilobecquerel per milliliter of the mixture. On the other hand, in some embodiments, the radium concentration is less than 1 megabecquerel per milliliter of the mixture, less than 700 kilobecquerel per milliliter of the mixture, less than 500 kilobecquerel per milliliter of the mixture, or even less than 400 kilobecquerel per milliliter of the mixture. The radium activity used optionally depends on the specific tumor type, according to the required biological effective dose (BED) of the tumor type and other properties of the tumor. Higher levels of activity are optionally used when a relatively large percentage of the mixture is expected to escape from the tumor.

The radium is optionally bounded to the agent. In some embodiments, after gelation, the radium is basically trapped in the mixture and the rate of radium atom release is less than 3% per day, less than 2% per day, or even less than 1% per day, during a period of at least 5 days or even at least 10 days from gelation. In some embodiments, the low leakage of radium from the mixture persists over a period of at least one half-life of the radium, at least 2 half-lives of the radium or even over at least three half-lives of the radium.

The diffusion of the radium, e.g., radium 224, in the mixture of the present embodiments is optionally very low, so that the radium does not escape the tumor in meaningful amounts. Optionally, the radium has a diffusion coefficient in the mixture of less than 10⁻¹² cm²/sec or even less than 2*10⁻¹³ cm²/sec.

On the other hand, upon radioactive decay of the radium, its daughter radionuclides (including radon and descendants thereof), have a substantial probability of leaving the mixture, for example with a release probability of at least 15%, at least 30% or even at least 40%. The radon daughter radionuclides of the radium radionuclide atoms optionally have a diffusion coefficient in the mixture greater than 10⁻⁷ cm²/sec.

In some embodiments, adding (106) the radium to the mixture is performed by first combining the radium to the agent and then adding the combined radium and agent to the vehicle. In other embodiments, the agent is first added to the vehicle and the radium is then added to the mixture of the agent and the vehicle. In further embodiments, the radium and the agent are first each placed in a separate solution, and these solutions are combined to form the mixture. In still other embodiments, the radium is first added to a calcium mixture or solution and then the combined radium and calcium are added to the mixture of the agent and the vehicle.

Optionally, adding (106) the radium is performed by adding a solution containing radium to the other components of the mixture. The solution containing radium may be generated using any suitable method known in the art, such as any of the methods described in PCT publication WO 2021/070029 “Wet preparation of Radiotherapy Sources”, the disclosure of which is incorporated herein by reference in its entirety. Alternatively or additionally, any of the methods described in Russian patent 2734429, U.S. Pat. No. 6,126,909 to Rotmensch et al., and/or U.S. Pat. No. 5,038,046 to Norman et al, the disclosures of which are incorporated herein by reference, are used to generate the solution with radium.

Alternatively, the radium is added (106) to the mixture, by placing the other components of the mixture in a flux of radium radionuclides. The flux is optionally generated by a flux generating surface source. For example, when the radionuclide is Ra-224, a flux thereof can be generated by a surface source of thorium-228 (Th-228). A surface source of Th-228 can be prepared, for example, by collecting Th-228 atoms emitted from a parent surface source of U-232. Such parent surface source can be prepared, for example, by spreading a thin layer of acid containing U-232 on a metal. Alternatively or additionally, the flux is generated using any of the methods described in US patent publication 2015/0104560 to Kelson et al., titled: “Method and Device for Radiotherapy”, the disclosure of which is incorporated herein by reference in its entirety.

Alternatively, dissolvable seeds carrying the alpha-emitter radionuclides are first generated, and the seeds are dissolved into other components of the mixture.

### Calcium

In some embodiments, the mixture includes calcium ions so that the gelation of the mixture within the tumor does not depend solely on calcium in the tumor.

Optionally, the calcium is added as free calcium ions. In some embodiments, a calcium compound is dissolved in a solution to create calcium ions, and the solution with the calcium ions is included in the mixture. The calcium compound optionally includes calcium chloride, although other calcium compounds may also be used, such as calcium nitrate, calcium acetate and/or calcium gluconate. In some embodiments, the calcium compound comprises calcium sulfate which was found by applicant to release the calcium relatively slowly and thus delays the hardening of the mixture. In other embodiments, in which very slow release of calcium is required, the calcium compound comprises calcium carbonate. In some embodiments, a mixture of a plurality of different calcium compounds is used.

In some embodiments, the calcium is added (108) to the mixture well before the injection of the mixture into the tumor. Optionally, in accordance with these embodiments, the calcium in the mixture is of a low concentration, which does not cause the mixture to turn into a gel on its own, but makes the gelation occur faster upon injection, and/or makes the viscosity of the mixture after gelation higher than when depending only on calcium in the tumor. In accordance with this option, the calcium is optionally provided in stand-alone calcium ions. In other embodiments, the calcium is included in calcium-loaded nanoparticles, such as liposomes. In some of these embodiments, the calcium-loaded nanoparticles are thermosensitive and set to release the calcium in body temperature or close to body temperature (e.g., from above 32° C., above 33° C., above 34° C.), so the agent turns into a gel after being inserted into the tumor. Alternatively or additionally, the calcium-loaded nanoparticles are designed to release their calcium upon injection due to other reasons, such as pH differences and/or due to a specific type of radiation, such as ultraviolet light and/or ultrasound. In still other embodiments in which calcium is added (108) to the mixture sufficiently before the injection into the patient, the added calcium causes gelation to occur before the injection and the mixture is injected in a gel form.

In other embodiments, the calcium is added (108) to the mixture shortly before the injection of the mixture into the tumor, and causes complete gelation or solidification only after several seconds or minutes, so that gelation and/or solidification of the mixture occurs only after injection.

Optionally, the calcium is added (108) to the mixture only after the radium is added (106) and bounded to the agent, such that the calcium does not interfere with bounding the radium to the agent. Alternatively, the calcium is added (108) to the mixture before the radium is added (106). In accordance with this alternative, the calcium in the mixture is optionally less than covers the entire agent, so as to allow the radium to bound to the agent.

The concentration of calcium in the mixture before injection governs the percentage of radium that remains in the tumor over time. A low concentration of calcium leads to a mixture that has a low viscosity, and hence higher levels of radium leave the tumor before radioactive decay. A high concentration of calcium may lead to too high a viscosity of the mixture at the time of injection to the tumor, such that the mixture is not injectable or does not disperse properly within the tumor. In some embodiments, the amount of calcium added is sufficient to form chunks in the gel. These chunks reduce the chances of the radium from leaving the tumor.

Optionally, the calcium is at least 0.1%, at least 0.3%, at least 0.5%, at least 0.8%, at least 1.5% or even at least 2.5% of the mixture in weight/weight (w/w). In some embodiments, the calcium is less than 10%, less than 8%, less than 6%, less than 5%, or even less than 4% of the mixture w/w. In some embodiments, for example embodiments in which the calcium in the mixture is not intended to cause the mixture to turn into a gel on its own, the calcium is added (108) at a concentration of at least 1 millimolar (mM), at least 2 mM, at least 4 mM, at least 5 mM, at least 6 mM, at least 8 mM or even at least 15 mM, but at a concentration of less than 60 mM, less than 50 mM, less than 30 mM, less than 26 mM, or even less than 23 mM. In some embodiments, the calcium is added (108) at a concentration of less than 15 mM, or even less than 12 mM. In some embodiments, 2 mM, 5 mM and 10 mM calcium are used. In other embodiments, for example embodiments in which the mixture is intended to be delivered to the treatment site as a gel, the calcium in the mixture is of a concentration of at least 50 millimolar, at least 75 millimolar, or even at least 100 millimolar.

Alternatively, the mixture does not include calcium, and the agent turns into a hydrogel within the tumor by collecting endogenous calcium present in the tumor after administering the mixture or by collecting calcium injected to the tumor separately, before, concurrently with or after injecting the mixture. Optionally, the calcium is injected to the tumor before, during and/or after injection of the mixture, through a separate needle, different from the needle used for injection of the mixture. In some embodiments, the calcium is injected concurrently with the mixture, for example using a split needle. The split needle is optionally designed to allow separate delivery of the calcium and the mixture to the tumor in a manner that the calcium and mixture come in contact only inside the tumor, so that the hardening of the mixture does not begin prematurely. Alternatively or additionally, the mixture is administered using a syringe controller which controls the rate of injection and/or retraction. Further alternatively or additionally, the mixture is administered using a needle having a plurality of apertures distributed circumferentially, so that the mixture is distributed around the needle during injection. In some embodiments, the mixture is designed to have a low pH value in order to extract calcium from surrounding tissue.

In still other embodiments, instead of using calcium to induce gelation of the agent, any other suitable component which hardens the agent is used, such as any bio-compatible element of the elements mentioned as inducing gelation of alginate in Hu et al., “Ions-induced gelation of alginate: Mechanisms and Applications”, International Journal of Biological Macromolecules, the disclosure of which is incorporated herein by reference. For example, instead of using calcium for gelation, magnesium and/or barium are used to induce the gelation. In some embodiments, calcium is used in addition to barium and/or magnesium to induce the gelation. In some embodiments, the mixture further includes chitosan which is expected to enhance the coupling of the mixture to the tissue of the tumor and thus reduce radium leakage.

### Contrast Agent

Optionally, the mixture includes a contrast material, such as gold, titanium, titanium oxide, zirconium oxide, silicon oxide, and/or any other suitable metal, which clearly appear in an imaging modality. In some embodiments, the contrast material comprises radionuclides other than radium, which emit, alpha, beta and/or gamma radiation to be used primarily for imaging. The contrast material is optionally provided as nanoparticles (e.g., gold nanoparticles). Alternatively or additionally, any other contrast material known in the art may be used, such as gadolinium (e.g., Gd³⁺) and/or methemoglobin (Met-Hb) as described in Marco L. H. Gruwel, et al., “Magnetic resonance imaging tracking of alginate beads used for drug delivery of growth factors at sites of cardiac damage”, Magnetic Resonance Imaging, Volume 27, Issue 7, 2009. In other embodiments, alternatively or additionally, the contrast material comprises indium-111. In still other embodiments, the contrast material comprises microbubbles which are visible at least using an ultrasound imaging modality. In some embodiments, the contrast material is selected as one that in addition to being seen by one or more imaging modality, serves to transform the agent into a gel in a manner similar to calcium, or to a lesser extent. For example, the contrast material may include barium. In some of these embodiments, the amount of calcium provided with the mixture or at a later stage is reduced.

Optionally, after injecting the mixture into a tumor, the tumor may be imaged to determine a layout of the mixture in the tumor and whether further injection of the mixture is required. In some embodiments in which the agent is included in small particles, the contrast material is included in the small particles. In some embodiments, the contrast material is mixed with the agent and the small particles are formed from the mixture of the contrast material and the agent. Alternatively or additionally, the small particles include a metal base, e.g., a gold base, coated by the agent, or by the agent with the inert excipient.

### Further Components

In some embodiments in which the radium is included in small particles, the small particles are targeted, by adding antibodies thereto. Optionally, additional materials are added in order to help in linking to antibodies for targeting the particles to the tumor.

Optionally, in order to prevent sodium ions in the patient from detaching the calcium formed bonds generating the gel, the mixture includes aldehyde, which prevents the sodium from detaching the bonds of the hydrogel.

The one or more additional materials includes, for example, any of the materials listed in Andrea Dodero et al., “An Up-to-Date Review on Alginate Nanoparticles and Nanofibers for Biomedical and Pharmaceutical Applications”, 2021, available at https://doi.org/10.1002/admi.202100809, the disclosure of which is incorporated herein by reference.

In some embodiments, the mixture further includes an emulsifier. The emulsifier is optionally provided along with an oil. The oil is optionally provided in an amount of at least three times the emulsifier, at least four times the emulsifier or even at least five times the emulsifier. In some embodiments, the emulsifier to oil ratio is 15:85. Optionally, the emulsifier is a non-specific surface active emulsifier, such as mannide-monooleate, a polysorbate and/or a hydrocolloid. Alternatively or additionally, the emulsifier comprises Tween80 and/or Span™ 85. In other embodiments, the emulsifier comprises lecithin, carrageenan, mono- and diglycerides, carboxymethylcellulose, sodium lauryl sulfate (SLS), benzalkonium chloride, cetearyl alcohol, stearic acid, glyceryl stearate and/or ceteareth-20.

The oil optionally comprises at least 66%, at least 70% or even at least 74% of the combination of the emulsifier and oil. In some embodiments, the oil constitutes less than 90%, less than 85%, less than 80% or even less than 77% of the emulsion administered to the patient. For example, in one embodiment, the oil forms 75% of the combination of the emulsifier and oil.

In some embodiments, the oil includes a mineral oil such as paraffin. In other embodiments, the oil is a non-mineral oil, such as squalene.

In some embodiments, the emulsifier and oil are taken as a known emulsifier and oil combination, such as Incomplete Freund's Adjuvant (IFA) or Complete Freund's Adjuvant (CFA). Other combinations that are used in some embodiments include MF59, which is formed of squalene (4.3%) in citric acid buffer with stabilizing nonionic surfactants Tween 80 (0.5%) and Span 85 (0.5%), AS03 formed of squalene, alpha-tocopherol (vitamin E), and polysorbate 80, Montanide ISA 51 formed of mineral oil (Drakeol 6 VR) and a surfactant from the mannide monooleate family and/or Montanide ISA 720 formed of a non-mineral oil (squalene) and a surfactant from the mannide monooleate family. The combinations of emulsifier and oil were found to encapsulate the mixture in a manner which increases the retention of the radium and thus increases the time the radium remains in the treated tumor. This, in turn, increases the tumor destruction effect and possibly induces a systemic immune effect.

The combination of the emulsifier and oil optionally forms at least 50% of the mixture used to treat the patient, and in some embodiments is at least 65%, at least 75% or even at least 80% of the mixture used to treat the patient. Optionally, the combination of the emulsifier and oil is less than 90% of the mixture used to treat the patient, less than 85% or even less than 83% of the mixture used to treat the patient. Generally, when the agent has a higher molecular weight (MW) and/or higher concentration, the combination of the emulsifier and oil forms a larger portion of the mixture administered to the patient. In some embodiments, for a solution of amount Y of a concentration X % of the agent in the solution, the combination of the emulsifier and oil is about [Y*(X−1)]±25%. Thus, for an agent solution of 2% agent, the combination of the emulsifier and oil is of about the same amount as the agent solution, for example between 75%-125% of the agent solution. For an agent solution of 4% agent, the combination of the emulsifier and oil is optionally between 275%-325% of the agent solution. In one embodiment, the mixture includes 4 parts of a water-based solution with 2% alginate and radium radionuclides, 3 parts oil and 1 part of an emulsifier (e.g., mannide-monooleate). In a second embodiment, the mixture includes 3 parts of a water-based solution with 4% alginate and radium radionuclides, 4 parts oil and 2 parts of an emulsifier.

The combination of the emulsifier and oil may be added to the mixture of any of the above embodiments, including those with and without calcium or other elements which turn the agent into a gel, and mixtures including drugs for slow release. While the combination of the emulsifier and oil may be used with any of the mixture embodiments described above, the mixtures which are emulsions optionally have a relatively low agent content, for example less than 4%, less than 3% or even less than 2% of the mixture in weight/weight. Similarly, the emulsion mixture optionally includes an agent with a low molecular weight (mW) of less than 125 kDa, less than 100 kDa, less than 75 kDa or even less than 60 kDa.

Optionally, the combination of the emulsifier and oil is added to the mixture after the radium and agent are mixed together, so that the emulsifier and/or oil do not interfere with the coupling of the radium to the agent. In some embodiments, the mixing of the components of the mixture is performed directly in a syringe or other medical tool used for administering (e.g., injecting) the mixture to the patient. In such cases, the emulsifier and oil, together with further optional components (e.g. immunoadjuvant, immunotherapy) if included, are loaded into the syringe separately from the water-based solution comprising the agent and radium, at a suitable volume relation (e.g. 1:1). Then, the components in the syringe are mixed inside the syringe, for example by attaching the syringe to a vortex or other rotating instrument. Mixing the ingredients of the mixture in the syringe used to administer the mixture avoids the need to transfer the mixture, with its viscosity and activity to a different vessel.

Furthermore, the combination of the emulsifier and oil is optionally the last ingredient added to the mixture, possibly followed only by addition of a diluent. After adding the emulsifier, the mixture is optionally shaken at a high speed, for example by a Vortex mixer. The shaking is optionally performed for at least 10 minutes, at least 20 minutes, at least 30 minutes, at least 45 minutes or even at least 1 hour. The shaking may be performed before the mixture is placed in a syringe used to administer the mixture to the patient, or may be performed when the mixture is already in the syringe. In some embodiments the mixing of the mixture with the emulsifier is carried out in a syringe system designed for mixing, for example in a system comprising two syringes connected by a luer-lock connector. In some embodiments, a Tuberculin needle is used to administer the mixture.

Alternatively or additionally, the mixture further includes a calcium chelator, such as Ethylenediaminetetraacetic acid (EDTA) and/or dodecane tetraacetic acid (DOTA, also known as tetraxetan), which reduce release of calcium and/or radium from the mixture. The calcium chelator included in the mixture optionally includes a number of molecules equal to at least 80%, at least 100%, at least 150% or even at least 180% of the number of calcium molecules provided in the treatment. In some embodiments, the number of molecules of the calcium chelator provided with the mixture is less than 4 times, less than 3 times, less than 2.5 times or even less than 2.2 times the number of calcium molecules provided in the treatment. In some embodiments, the number of molecules of the calcium chelator provided with the mixture is twice the number of calcium molecules provided in the treatment.

In some embodiments, the one or more additional materials do not substantially prevent diffusion of radium daughters (including daughters of daughters down a decay chain). That is, the one or more additional materials are optionally chosen as materials which do not couple to and/or block diffusion of progeny of radon, such as lead.

Further alternatively or additionally, the vehicle comprises an in-situ gelling polymer, which is supplied in a sol form at room temperature and upon injection into a patient transforms into a gel state, in response to a change in temperature, pH and/or ionic composition. For example, any suitable in-situ gelling polymer described in Kouchak M. In situ gelling systems for drug delivery, Jundishapur J Nat Pharm Prod. 2014 Jun. 1; 9(3):e20126. doi: 10.17795/jjnpp-20126. PMID: 25237648; PMCID: PMC4165193 and/or in Xian Jun Loh, “In-Situ Gelling Polymers, for Biomedical Applications”, 2014, the disclosures of which are incorporated herein by reference.

In some embodiments, the mixture further includes disodium phosphate, as described in “Factors Affecting Calcium Phosphate Mineralization Within Bulk Alginate Hydrogels”, Journal of Polymer Research 26(12), December 2019, the disclosure of which is incorporated herein by reference in its entirety.

In still other embodiments, the mixture includes materials which increase the stability of the agent, such as gelatin, Chitosan, and/or polymer phosphate. Optionally, the materials which increase the stability or included in an amount of at least 50%, at least 80% or even at least 95% of the mixture of the agent and the vehicle. In some embodiments, the weight of the materials which increase the stability is not more than 150%, not more than 125% or even not more than 110% of the weight of the mixture of the agent and the vehicle. In some embodiments, the mixture includes liposomes, which are believed to further the effect of fixing the mixture in the tumor or its surroundings.

Combination with Other Drugs

In some embodiments, in addition to alpha-emitter radium radionuclides, the mixture includes, one or more drugs. Optionally, the mixture operates as a slow release mechanism of the one or more drugs. The one or more drugs may be any drugs suitable for treatment of the patient, for example drugs known to have a positive synergy with alpha-emitter radiation treatment. In some embodiments, the one or more drugs include a substance which activates cytoplasmatic sensors for intracellular pathogen in the tumor, as described for example in PCT publication WO 2020/089819, titled: “Intratumoral Alpha-Emitter Radiation and Activation of Cytoplasmatic Sensors For Intracellular Pathogen”, the disclosure of which is incorporated herein by reference in its entirety. Alternatively or additionally, the one or more drugs include an immune checkpoint regulator, such as immune checkpoint inhibitors (e.g., aPD-1, aCTLA4, aLAG3, aTim3, aTIGIT), for example as described in PCT application PCT/IB2022/055680, titled: “Intratumoral Alpha-Emitter Radiation in combination with Checkpoint Regulators”, the disclosure of which is incorporated herein by reference in its entirety. Further alternatively or additionally, the one or more drugs include a vasculature inhibitor, such as described in PCT application PCT/IB2022/055679, titled: “Intratumoral Alpha-Emitter Radiation in combination with Vasculature Inhibitors”, the disclosure of which is incorporated herein by reference in its entirety. In some embodiments, the one or more drugs included in the mixture comprise immunoadjuvants, such as TLR agonists, or viral DNA/RNA mimics, such as PolyICLC, BO112, CpG and/or PolyIC. Alternatively or additionally, the immunoadjuvants include CpG-1018, Aluminum salt, monophosphoryl lipid A (MPL) or combinations thereof, such as AS04, formed of aluminum salts and 3′-O-deacylated monophosphoryl lipid A (MPL).

Alternatively or additionally, the one or more drugs included in the mixture comprise antibodies such as antiangiogenic agents and/or immune blockades. Further alternatively or additionally, the one or more drugs comprise ligands to pattern recognition receptors (PRRs Ligands, e.g. the ligands of TLR, NOD, RLR, CLR, cGAS, STING, AhR, ALPK1, Multi-PRR, Conjugatable PRR; Inflammasome Inducers or NF-κB & NFAT Activators). In some embodiments, the one or more drugs include a Mycobacterium or its modified form. The Mycobacterium may include, for example, Mycobacterium tuberculosis or Mycobacterium bovis. The term Mycobacterium in the present specification and claims is to be understood as including both a live Mycobacterium, a modified and/or weakened Mycobacterium and a dead Mycobacterium.

Alternatively or additionally, the one or more drugs include any other suitable drug for chemotherapy, immunotherapy, Gene therapy, targeted therapy and/or antiangiogenic treatment. The one or more drugs may be dispersed throughout the mixture and/or may be placed in the small particles. In some embodiments, the one or more drugs are provided in parallel to the mixture, not in the mixture.

### Combined Treatment

Optionally the mixture can be injected to a patient in combination with one or more other anticancer treatments such as radiation, immunotherapy, targeted therapy, chemotherapy and surgery. Optionally these treatments synergize with alpha-emitters (refer to the previous patents). Optionally the other anticancer treatments be given before, concurrently or after the injection of the mixture.

### Experiment

Applicant has performed an experiment in which a radium water-based solution was mixed with a sodium alginate solution such that the final concentration of sodium alginate in the solution was 4% w/w. The resultant solution was mixed with a calcium chloride water-based solution in the concentration of 1% w/w. The resultant hydrogel was then placed in fetal bovine serum. The serum was replaced by a clean serum solution every 3 days for 4 times, and the replaced serum was subjected to activity measurements that characterized the Probability of Pb-212 release from the gel to the serum (%) and the rate of radium leakage from gel per day (%), for each timepoint.

The results for each timepoint are presented in the following table:

| | Probability of Pb-212 | Ra-224 | Ra-224 |
|---|---|---|---|
| | release from | leakage from | leakage from |
| Day | gel, % | gel, % | gel, per day % |
| day 3 | 41% | 5% | 1.7% |
| day 6 | 46% | 3% | 1.0% |
| day 9 | 30% | 3% | 1.0% |
| day 12 | 24% | 2% | 0.7% |

The experiment shows that Ra-224 molecules are relatively highly fixated to alginate-based hydrogel, while radium daughter atoms are released from the gel to the outer liquid environment, in substantial amounts. Other gels may allow leakage of larger percentages of radium or have a much lower release rate of daughter radionuclides.

In another experiment performed by applicant, a mixture including 2% alginate by volume and 2 millimolar calcium was injected to tumors in mice. The mixture injected to each tumor had a volume of about 50% of the tumor. The activity of the radium in the mixture was about 2 microcurie for a tumor of about 20-150 millimeter cube and the net activity in the tumor was about 20 Kilobecquerel as measured by a gamma counter when dissecting several of the experiment mice after several days.

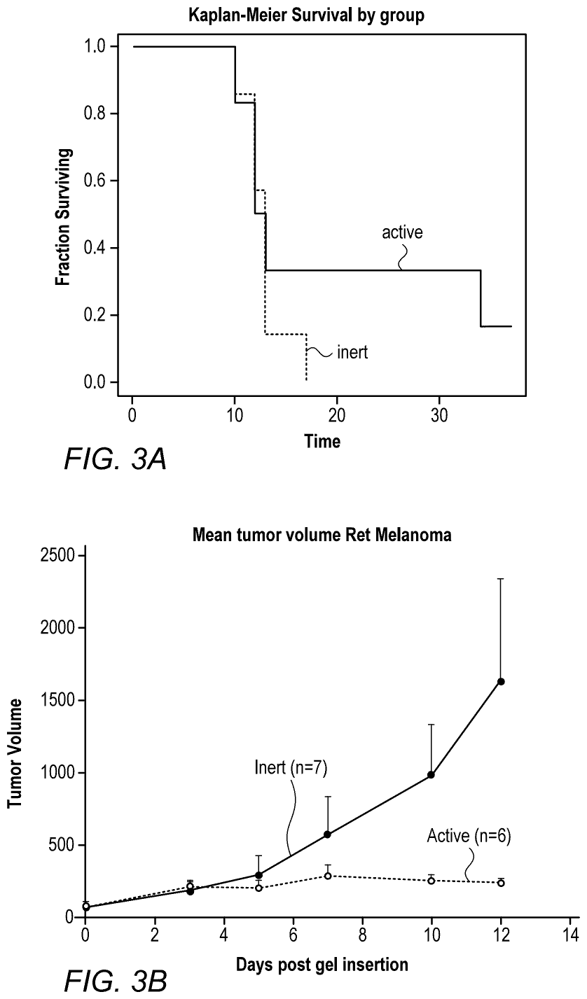

FIGS. 3A and 3B show results of the experiment. FIG. 3A shows the percentage of mice that survived as a function of days from treatment. Mice that received the inert treatment all died by 18 days after the treatment, while a third of the mice that were injected with alginate carrying active radium survived over 30 days.

FIG. 3B shows the tumor size as a function of the number of days after treatment. As can be seen, the tumors treated with alginate carrying active radium do not grow substantially, while those in the control group become very large.

Further experiments compared the radium retention after 2-3 days from injection of different alginate solutions. For a solution of 2% alginate, an average 7% retention rate of radium was measured. For a solution of 2% alginate to which IFA was added at a relation of 1:1 with the alginate solution, an average 24% retention rate of radium was measured. For a solution of 4% alginate, an average 20% retention rate of radium was measured. For a solution of 5% alginate, an average 26% retention rate of radium was measured. For a solution of 4% alginate with 10 mM calcium, an average 35% retention rate of radium was measured.

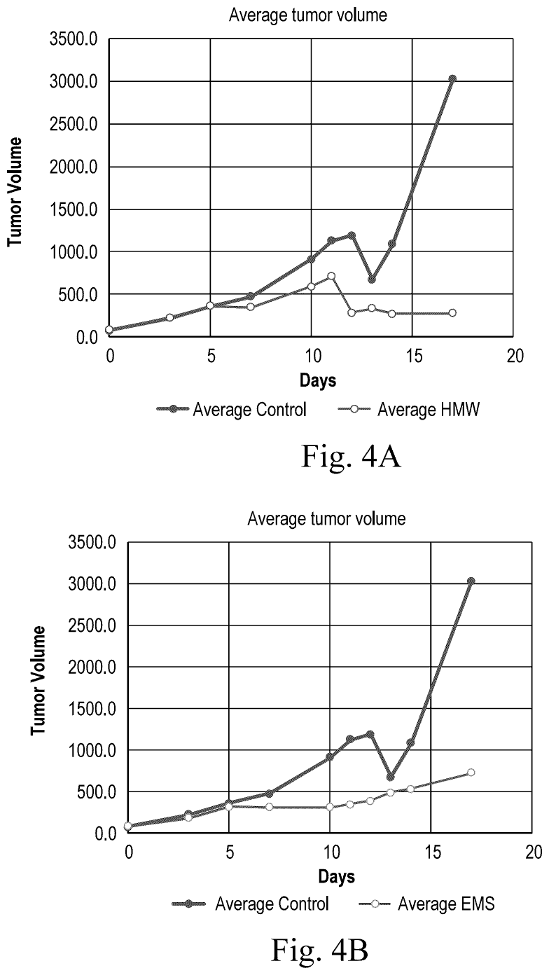

FIG. 4A shows the average tumor size as a function of the number of days after treatment, for mice treated with a 4% PRONOVA HMW alginate solution compared to mice in a control group.

FIG. 4B shows the average tumor size as a function of the number of days after treatment, for mice treated with an emulsion and alginate solution compared to mice in a control group. It is noted that the activity of the emulsion and alginate solution is one quarter of the activity of the alginate solution of FIG. 4A.

### Usage

Exemplary tumors that can be treated by the mixture include tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma, hepatoma, heterohybridoma, heteromyeloma, histiocytoma, hybridoma (e.g., B cell), hypernephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, medullary thyroid, multiple meningioma, endocrine neoplasia, myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma. In some embodiments, the mixture is used to treat eye cancer, e.g., uveal melanoma.

### Administration

FIG. 2 is a flowchart of acts performed in treatment of a tumor, in accordance with an embodiment of the present invention. The method (200) optionally includes identifying (202) a tumor or other tissue requiring treatment and estimating (204) an amount of the mixture required to be injected and/or selecting (206) the precise composition of the mixture to be injected to the identified tumor. Thereafter, the mixture is injected (208) to the tumor. In some embodiments, calcium is injected (210) to the tumor before, during and/or after injection of the mixture. Optionally, a medical image of the tumor is acquired and analyzed (212), to determine whether the entire volume of the tumor is covered by the mixture. If necessary, a further injection (208) of the mixture to uncovered areas of the tumor is carried out.

As to identifying (202) the tumor, the tumor may be a primary tumor or a metastatic tumor. The use of an injectable mixture is believed to allow treatment of a larger number of small metastatic tumors in a single patient, relative to implantation of solid seeds.

Referring to estimating (204) of the amount of the mixture to be injected, the amount is optionally selected proportionally to the size of the tumor. In some embodiments, the amount of the mixture injected to a tumor is at least 3%, at least 5%, at least 10%, at least 25% or even at least 30% of the volume of the tumor. The injected mixture is optionally of a volume of less than 60%, less than 50%, less than 25%, less than 20% or even less than 15% of the volume of the tumor.

In some embodiments the mixture is injectable and/or non-solid. In some of these embodiments, the mixture is injected as a liquid solution. Optionally, the mixture when injected has a viscosity lower than 25 millipascal seconds, lower than 20 millipascal seconds, lower than 15 millipascal seconds, or even lower than 10 millipascal seconds. Alternatively, the mixture has a gel structure when being injected. At the time of injection, the mixture optionally has a viscosity of at least 20 cP, but less than 10,000 centipoise (cP), less than 5,000 cP or even less than 3,000 cP, so as to allow easy injection to a tumor.

The mixture optionally has a much higher viscosity when smeared on a tumor. In such embodiments, the viscosity is optionally greater than 20,000 cP or even greater than 50,000 cP. Furthermore, in some cases the mixture is administered in a rigid and/or non-liquid state. For example, the mixture is casted in the form of a seed which is implanted in a tumor, or is formed as a sheet and placed adjacent a tumor.

Optionally, inside the tumor after being injected, the viscosity of the mixture increases, and possibly even solidifies. Optionally, the viscosity of the mixture is adjusted by changing its pH. In some embodiments, the mixture is thermosensitive. At room temperature (e.g., 21° C.) or lower, the mixture optionally has a viscosity of less than 10,000 centipoise (cP), less than 5,000 cP or even less than 3,000 cP, while in body temperature the mixture has a high viscosity of at least 75,000 cP, at least 85,000 cP, at least 90,000 cP or even at least 95,000 cP. Further alternatively, the viscosity of the mixture increases in the tumor due to its exposure to endogenous calcium ions. In other embodiments, however, at the time of injection the mixture has a high viscosity of at least 20,000 cP, at least 40,000 cP or even at least 70,000 cP, so that the mixture substantially remains close to the point in which it was injected.

In other embodiments, the mixture solidifies before being administered and is introduced into the tumor in the form of flexible or even solid seeds of any suitable shape, such as spheres, thin strands or bricks. The seeds optionally have a width of at least 100 microns, at least 200 microns, at least 300 microns or even at least 400 microns. The seeds optionally have a largest dimension of less than 5 millimeters, less than 2 millimeters, less than 1 millimeter or even less than 0.8 millimeters. Optionally the seeds are produced from the agent that turns into a hydrogel and calcium in a first stage, which may be carried out a long time (e.g., at least two weeks, at least a month or even at least two months) before treatment of the patient. In a second stage, carried out shortly (e.g., less than 48 hours, less than 24 hours or even less than 12 hours) before the treatment radium is added to the seeds, for example by placing the seeds in a radium solution.

In some embodiments, the solid seeds are placed on a surface requiring treatment, for example a wall of a cavity. In other embodiments, the solid seeds are implanted in a tumor, for example by introducing a needle filled with the solid seeds into the tumor and retracting the needle such that the solid seeds remain in the tumor.

In still other embodiments, the mixture is included as an outer layer on a seed, catheter or needle inserted into the tumor. Optionally in these embodiments, the seed is covered by a protective layer which protects the mixture from being removed from the seed before insertion into the tumor.

As to selecting (206) the precise composition of the mixture to be injected, in some embodiments, the composition of the mixture and/or the amount of separately injected materials is selected responsive to the type of the tumor (e.g., squamous cell carcinoma, melanoma, glioblastoma, etc.), in order to maximize the percent of radium retained in the tumor. Applicant has found that different types of tumors have a different optimal amount of calcium to be provided with the mixture, to maximize the percent of radium retained in the tumor. For example, when injecting the mixture to closed organs which retain liquids, such as in the eyeball (uveal melanoma) or close to the muscle (e.g., fibrosarcoma), lower amounts of calcium and/or any other gelation substance are included in a lower amount, or not included in the mixture at all.

In some embodiments, the mixture is injected (208) to a core of the tumor. Alternatively or additionally, the mixture is injected to margins of the tumor and/or adjacent the tumor outside of the tumor, for example when treating remnants of a tumor after removal by surgery. In some embodiments, the mixture is injected throughout the tumor. In some embodiments, a needle tip through which the mixture is injected is moved, e.g., retracted, during the injection in order to increase the surface area of the mixture in the tumor, and thus increase the volume of the tumor effected by the radium. The mixture is optionally injected to the tumor in a single insertion of a needle. Alternatively, the mixture is injected to the tumor in a plurality of needle insertions at different times and/or in different areas of the tumor, in order to increase the volume of the tumor covered by the injected mixture and/or to treat tumor cells that were not exposed to enough radiation in previous injections.

In other embodiments, rather than injecting the agent solution, the radium and/or calcium, other administration methods are used. For example, in some embodiments, the mixture is smeared or spread on a surface requiring treatment, for example on a surface of a tumor (e.g., an external skin cancer tumor) and/or on a surface of a cavity from which a tumor was removed by surgery, ablation or any other suitable method. For example, the cavity may be from removal of a primary or metastatic tumor in the liver or brain. In some of these embodiments, a protective sheet may be placed on the smeared mixture in order to prevent damage to healthy tissue. Particularly, when treating a blood vessel, such as an artery, from which a tumor was removed (e.g., SMA when a tumor is removed from the pancreatic head), the mixture with radium is placed around the blood vessel in the area from which the tumor was removed and a protective sheet is placed on the tumor around the blood vessel on the mixture. The protective sheet is optionally fixed in place for example by a fixation ring which can be buckled to tightly surround the protective sheet.

While the above description relates primarily to administering the mixture to a tumor, the mixture may be administered in some embodiments outside a tumor or without relation to a specific tumor. For example, for a patient with multiple metastases and/or cancerous cells floating and/or dispersing in an internal space (e.g., stomach, pelvis, bladder, uterus, chest, thoracic cavity, abdominal cavity), the mixture may be injected into the internal space to spread throughout the internal space. In some embodiments, the mixture is administered next to a tumor requiring treatment, not necessarily in the tumor.

In some embodiments, the mixture is introduced into the cavity in a manner which fills the cavity. In other embodiments, the injected mixture does not fill the cavity and a balloon is introduced to the cavity and inflated therein to press the mixture onto the surfaces of the cavity. Alternatively, a balloon is inflated in the cavity before injecting the mixture and the mixture is injected after inflating the balloon to fill the area in the cavity not filled by the balloon. Calcium is introduced in some embodiments into the cavity, after the balloon and the mixture are in place to harden the mixture. Optionally, the balloon is removed only after the mixture sufficiently hardens. In some embodiments, after removing the balloon, an inert mixture of alginate or other suitable material is introduced to the cavity to fill the tumor.

In some embodiments, before administering the mixture to a cavity or other surface near a tumor, a bag is placed near the surface and the mixture is injected into the bag. For example, the bag may be placed in the cavity during an operation for removal of a tumor in which the cavity is created. The bag is optionally formed of a polymer which is permeable to radon, such as a silicone rubber (e.g., polydimethylsiloxane (PDMS)) or polypropylene. The bag has a thin wall at least on its side which faces the surface to be treated, so that radon leaving the mixture will be able to pass through the bag wall and treat the surface tissue. In some embodiments, the opposite side of the bag is thicker than the side facing the treated surface, and sufficiently thick to prevent release of radon. Optionally, the treatment side of the bag has a thickness of less than 100 microns, less than 80 microns or even less than 60 microns. The opposite side of the bag optionally has a thickness of at least 100 microns or even at least 150 microns. A balloon or inert filler is used, in some embodiments, to urge the bag or gel slices against the surface to be treated.

The thickness of the mixture layer placed on the surface to be treated optionally depends on the type of the tumor and/or the depth of the tumor below the surface. For deep tumors, a thicker layer of the mixture is desired, in order to increase the amount of beta radiation emitted by the radium.

Alternatively to injecting to the tumor, the mixture is injected to the patient's blood circulation, for example when the mixture includes targeted small particles.

In some embodiments, the mixture is injected to a vasculature next to the tumor in a manner which blocks and/or closes small blood vessels near or in the tumor. In embodiments using targeted nanoparticles, the mixture may additionally or alternatively be administered intravenously.

As discussed above, not all embodiments involve injecting (210) calcium. In some embodiments in which calcium is injected (210), the calcium is injected to the tumor before, during and/or after injection of the mixture through a separate needle from the needle used for injection (208) of the mixture. The separate needles may access the tumor through a same aperture in the patient or through different apertures. In some embodiments, the calcium is injected (210) concurrently with the mixture, for example using a split needle. Alternatively, the mixture and the calcium are loaded into the same needle and are injected one after the other into the tumor. Optionally, the calcium is loaded to the needle more distally and is injected first into the tumor. Alternatively, the mixture is loaded to the needle more distally and is injected before the calcium.

Optionally, the separate administration of calcium is injected to the tumor in a single injection. Alternatively, calcium is injected to the tumor in a plurality of injection sessions, for example at least two, at least three or even at least four injection sessions. Optionally, the injection sessions are separated from the injection (208) of the mixture by at least 1 hour, at least 3 hours, at least 6 hours, at least 12 hours or even by at least 24 hours. The multiple injections at separate times are believed to better achieve gelation of the mixture and reduce the percentage of the mixture that leaves the tumor. It is believed that when a large amount of the agent is mixed with a large amount of calcium in a tumor, not all the agent interacts with the calcium and turns into a gel. The excess calcium that did not participate in the gelation escapes the tumor and a further dose of calcium is required for gelation of the portion of the agent that did not undergo gelation.

It is noted that the separate administration of calcium may be performed even in cases in which the mixture includes calcium, in order to increase the amount of calcium administered with the agent. In some embodiments in accordance with this option, most of the calcium administered to the tumor (e.g., at least 50%, at least 60% or even at least 75%) is included in the mixture with the agent. In other embodiments, most of the calcium administered to the tumor (e.g., at least 60%, at least 70% or even at least 80%) is administered separately from the mixture including the agent. These embodiments are used to avoid hardening of the mixture before injection to the tumor.

Injecting the calcium before the mixture increases the percentage of the mixture that turns into a gel before it leaves the tumor, as the calcium required to induce the gelation is already in the tumor when the mixture is injected. Concurrent injection of the mixture and the radium allows simpler operation by the medical practitioner performing the injection, while inducing immediate mixing of the agent in the mixture and the calcium.

In the above description, the agent and the radium are mixed together into the injected solution, before the injection. In other embodiments, a solution including the agent is injected to the tumor separately from administration of the radium radionuclides, before, concurrently with or after the injection of the agent. In some of these embodiments, the radium radionuclides are administered by injecting a radium solution into the tumor. Possibly, the solution including the radium also includes calcium. Alternatively, the radium radionuclides are administered by insertion of one or more sources which carry free radium, into the tumor. The one or more sources are optionally designed to allow the free radium to leave the sources, after implantation of the one or more sources in the tumor. The sources, optionally, are biodegradable after implantation, so that the free radium is released into the tumor. Alternatively, the radium on the sources is attached loosely so that the radium is released from the source upon contact with tumor tissue and/or due to the temperature in the tumor. The radium released from the seeds is caught by the solution including the agent, and is thus prevented from escaping the tumor.

The separate administration of the radium allows producing the agent solution a longer time before the treatment and storing the agent solution for longer periods. In some embodiments, the agent solution is injected before the radium is administered, for example at least 5 seconds, at least 15 seconds, at least 45 seconds or even at least 90 seconds before the administration of the radium to the tumor. The early injection of the agent allows the agent time to settle in the tumor, before the radium is administered. Optionally, however, the radium is administered within less than 1 hour, less than 20 minutes or even less than 10 minutes from the injection of the agent solution. Alternatively, the agent solution is injected after administering radium to the tumor. Optionally, in this alternative, the agent solution is injected within less than 30 seconds, less than 15 seconds or even less than 5 seconds from the administering of the radium, so that the radium does not escape before the agent solution which provides long term fixation of the radium in the tumor is injected. In some embodiments administering the radium by injecting a radium solution, the radium solution and the agent solution are injected using a same needle. Alternatively, the radium solution and the agent solution are injected from different needles. In other embodiments, the separate injection of the radium solution and the agent solution is performed concurrently from two different needles.

In embodiments in which the mixture with the agent is injected to the tumor before injecting the radium, the time between injecting the agent and injecting the radium is preferably sufficiently long, such that a large percentage of the agent that is expected to leak out of the tumor leaves the tumor before the radium is injected to the tumor. In some embodiments, the mixture with the agent is injected to the tumor at least an hour, at least three hours, at least 4 hours, at least six hours or even at least 12 hours before injecting the radium. Thus, the agent is expected to settle in the tumor and the percentage of radium leaving the tumor is expected to be relatively low. Optionally, the radium is injected to the tumor less than 48 hours, less than 36 hours, less than 24 hours or even less than 18 hours after the mixture with the agent is injected to the tumor, so as to use the agent before it deteriorates in the patient.

In embodiments in which the mixture with the agent is injected with the radium, the time between injecting the agent and injecting the calcium is optionally short, for example less than 30 minutes, less than 10 minutes, less than 5 minutes or even less than 1 minute, so that the hardening occurs as promptly as possible and the leakage of radium from the tumor is minimized.

In the method of FIG. 2, further injection (208) of the mixture is performed responsive to analysis (212) of medical images of the tumor. The acquiring of the images may be performed immediately after the injection and/or at later times, for example after several days (e.g., every 2 days or every 3 days). Alternatively or additionally, repeated injection may be performed periodically based on a predesigned plan. For example, repeated injections may be performed repeatedly separated by intervals of at least 24 hours, at least two days, at least five days or even at least a week. The separation intervals are optionally shorter than a month or even shorter than two weeks. The repeated injections may be performed, for example, every two days, every week or every two weeks. In some embodiments, when treating a patient with multiple tumors, the mixture is injected to a plurality of separate tumors of the patient in a single treatment session. Alternatively, the mixture is injected to each tumor at a separate time, so as not to introduce large levels of radioactivity concurrently.

In some embodiments, medical images are used in order to identify leakage of the mixture from the tumor.

The medical images may be of any suitable modality, such as MRI, CT and/or ultrasound.

### CONCLUSION

While the above description relates to a mixture of an agent which turns into a hydrogel by addition of calcium ions with alpha emitter radium isotopes, it is noted that the agent could be used with proper adaptations also with betta emitter radium, such as radium-225. Furthermore, the agent could be used to form a radiotherapy mixture with radionuclides of other isotopes of biocompatible elements which transform the agent into a gel, or otherwise bond to the agent, and have a half-life suitable for use in medical radiotherapy. Elements that transform the agent into a gel could be any of those described in the above mentioned article: Hu et al., “Ions-induced gelation of alginate: Mechanisms and Applications”, International Journal of Biological Macromolecules, including Ba²⁺, Cu²⁺, Sr²⁺, Fe²⁺, Zn²⁺, Mn²⁺, Al³⁺ and Fe³⁺ and elements having similar properties, such as elements in the columns of these elements in the periodic table. These other isotopes are optionally beta emitters, positron emitters and/or electron capturers. These other isotopes include, for example, isotopes of strontium (⁹⁰Sr and ⁸⁹Sr, ⁸⁵Sr), isotopes of calcium, copper-64 and/or gold-198. It is noted that the isotopes may be provided as free ions or in compounds which separate after being added to the mixture. It will be appreciated that the above-described methods and apparatus are to be interpreted as including apparatus for carrying out the methods and methods of using the apparatus. It should be understood that features and/or steps described with respect to one embodiment may sometimes be used with other embodiments and that not all embodiments of the invention have all of the features and/or steps shown in a particular figure or described with respect to one of the specific embodiments. Tasks are not necessarily performed in the exact order described.

It is noted that some of the above-described embodiments may include structure, acts or details of structures and acts that may not be essential to the invention and which are described as examples. Structure and acts described herein are replaceable by equivalents which perform the same function, even if the structure or acts are different, as known in the art. The embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art. Therefore, the scope of the invention is limited only by the elements and limitations as used in the claims, wherein the terms “comprise,” “include,” “have” and their conjugates, shall mean, when used in the claims, “including but not necessarily limited to.”

1. A method of generating an emulsion for treating a patient, comprising:
generating a water-based solution carrying an agent which turns into a hydrogel by addition of calcium ions;
adding radium radionuclides to the water-based solution in a concentration sufficient for radiotherapy; and
mixing the water-based solution with an emulsifier and an oil, after adding the radium to the water-based solution.

2. The method of claim 1, wherein the emulsifier with the oil are of a volume greater than the volume of the water-based solution.

3. The method of claim 1, wherein the agent which turns into a hydrogel by addition of calcium ions is between 0.5-4% of the water-based solution.

4. The method as in claim 3, wherein the agent which turns into a hydrogel by addition of calcium ions is between 1.2-2.4% of the water-based solution.

5. The method as in claim 1, wherein the emulsifier and oil form less than 80% of the emulsion.

6. The method as in claim 1, wherein the emulsifier and oil form at least 75% of the emulsion.

7. The method as in claim 1, wherein the emulsifier with the oil is Incomplete Freund's Adjuvant (IFA) or Complete Freund's Adjuvant (CFA).

8. The method as in claim 1, wherein the oil comprises squalene.

9. The method as in claim 1, wherein the mixture further comprises CpG-1018 or monophosphoryl lipid A (MPL).

10. The method as in claim 1, wherein the emulsion does not include targeting elements which make the emulsion suitable for targeted therapy.

11. The method as in claim 1, further comprising adding to the water-based solution an immune-checkpoint inhibitor.

12. The method as in claim 1, further comprising adding to the water-based solution an immunoadjuvant.

13. The method as in claim 1, further comprising adding a mycobacterium to the water-based solution.

14. The method as in claim 1, further comprising adding a DNA or RNA viral mimic to the water-based solution.

15. The method as in claim 1, further comprising adding a tumor antigen to the water-based solution.

16. The method as in claim 1, wherein the agent comprises alginate.

17. The method as in claim 1, wherein the radium radionuclides are radium-224 radionuclides.

18. The method as in claim 1, wherein the emulsion:
does not include calcium; or
does not include a sufficient amount of calcium for turning the agent into a hydrogel.

19. The method of claim 1, wherein generating the water-based solution comprises dispersing the agent homogenously in the water-based solution.

20. The method of claim 1, wherein mixing the water-based solution with an emulsifier and an oil is performed in a syringe suitable for injecting the emulsion to the patient.

21. A medicament for treating a tumor, comprising:
an agent which turns into a hydrogel by addition of calcium ions;
a water-based solution carrying the agent in a manner allowing injection of the medicament into a patient;
an emulsifier;
an oil; and
radium radionuclides bounded to the agent in a concentration sufficient to treat the tumor by radiotherapy.

22. The medicament as in claim 21, wherein the agent is dispersed homogenously in the water-based solution.

23. The medicament as in claim 21, wherein the medicament does not include targeting elements sufficient for targeted therapy of the medicament.

24. The medicament as in claim 21, further comprising a substance which regulates immune-checkpoints dispersed in the medicament.

25. The medicament as in claim 21, further comprising an immunoadjuvant.
